(19) United States
(12) Patent Application Publication
(10) Pub. No.: US 2025/0288295 A1
(43) Pub. Date: Sep. 18, 2025

(54) EXPANDABLE EMBOLIC IMPLANTS WITH POSITION-FIXING COVERS

(71) Applicant: Polyembo, LLC, Park City, UT (US)

(72) Inventors: Daniel J. Ashurst, Mead, CO (US); Shawn P. Fojtik, Park City, UT (US); Joseph R. Steele, Houston, TX (US)

(21) Appl. No.: 19/080,785

(22) Filed: Mar. 14, 2025

Related U.S. Application Data
(60) Provisional application No. 63/565,501, filed on Mar. 14, 2024.
(60) Provisional application No. 63/569,145, filed on Mar. 23, 2024.

(51) Int. Cl. A61B 17/12; A61B 17/00

(52) U.S. Cl. CPC A61B 17/12109; A61B 17/1204; A61B 17/12172; A61B 2017/00862; A61B 2017/00867; A61B 2017/12095

(57) ABSTRACT

An embolic implant includes an expandable section and a membrane over the expandable section. As the expandable section expands, the membrane also expands to define a boundary between an interior of at least a portion of the expandable section and an exterior of the expandable section. The membrane includes a primary opening to enable fluid to flow into the interior. The membrane may optionally include one or more secondary openings that prevent over pressurization of the membrane. The pressure within the membrane may be communicated by the membrane to surfaces against which the membrane is positioned, which may enable the membrane to at least partially hold the embolic implant in place.

### CROSS-REFERENCE TO RELATED APPLICATIONS

Claims for priority are made pursuant to 35 U.S.C. § 119 (e) to the Mar. 14, 2024 filing date of U.S. Provisional Patent Application No. 63/565,501, titled EXPANDABLE OCCLUSIVE DEVICES (“the '501 Provisional application”) and the Mar. 23, 2024 filing date of U.S. Provisional Patent Application No. 63/569,145, titled EXPANDABLE EMBOLIC DEVICES (“the '145 Provisional application”). The entire disclosures of the '501 Provisional Application and the '145 Provisional Application are hereby incorporated herein.

### TECHNICAL FIELD

This disclosure relates generally to embolic implants and, more specifically, to an embolic implant with a cover that fixes a position of the embolic implant at a desired location along a length of a vessel within a body of a subject, or facilitates securement of the embolic implant at the desired location along the length of the vessel. This disclosure also relates to methods for placing embolic implants.

### RELATED ART

Occlusive devices, including plugs and coils, are used to occupy blood vessels and voids within a subject's body. Occlusive devices may therapeutically and/or diagnostically slow or stop the flow of blood though blood vessels or occlude other voids within a subject's body. Occlusive devices may be used for a variety of purposes, including treating arteriovenous malformations, controlling bleeds, closing perforations, blocking aneurysms, devascularization and isolated treatment of tumors, and other conditions.

Occlusive devices are typically self-expanding devices designed to be constrained in a loading device, pushed through a tubular delivery device to a target location, where the occlusive device self-expands to occlude the target location. Existing occlusive devices may be manufactured from metal or polymer. The occlusive devices may occlude blood flow on their own, or they may be supplemented with other occlusive features.

Occlusive devices may be manufactured to form any of a number of different three-dimensional shapes when deployed. The tertiary shape of an occlusive device may enable it to serve as a primary occlusion or enable it to be used with other occlusive devices to occlude a vessel. For example, a first occlusive device may be anchored in place and other occlusive devices be packed behind the first occlusive device.

While existing occlusive devices are useful, their ability to remain in place is limited by their size and the extent to which their basic structures enable them to pack together. Occlusion of a blood vessel with conventional occlusive devices typically requires the placement of five or more of the conventional occlusive devices in proximity to each other within the blood vessel.

### SUMMARY

An embolic implant of this disclosure includes a proximal end, an expandable section, and a distal end, with the proximal end and the distal end being on opposite sides of the expandable section. The embolic implant may also include a membrane on the expandable section. The cover may include at least one opening located so as to be positionable against (i.e., to receive) fluid flowing (e.g., blood flow, etc.) toward the embolic implant.

The proximal end, expandable section, and distal end of the embolic implant may be formed from a tube. Without limitation, the tube may comprise a hypotube. In some embodiments, the hypotube may be formed from a metal or a metal alloy. For example, the hypotube may be formed from a shape memory material, such as a shape memory alloy (e.g., a nickel-titanium alloy, or nitinol, etc.). As another example, the hypotube may be defined from a stainless steel (e.g., an austenitic stainless steel, such as grade 304 stainless steel, grade 316 stainless steel, grade 316L stainless steel, etc.). In other embodiments, the hypotube may be formed from a suitable polymer (e.g., polyether ether ketone (PEEK), polyimide, polytetrafluoroethylene (PTFE), etc.).

The expandable section of the embolic implant may have an unexpanded arrangement, or a collapsed state, that facilitates introduction of the embolic implant into a body of a subject; for example, into a vessel or other space within the body of the subject. In embodiments where the expandable section is formed from a tube, the expandable section may assume or substantially assume the native shape of the tube while the expandable section is in its unexpanded arrangement.

The expandable section may be expandable from its unexpanded arrangement to an expanded arrangement, or an expanded state. The expandable section may self-expand or it may expand as a length-compressing force and/or a diameter-expanding force is applied to it. As the expandable section expands from its unexpanded arrangement to its expanded arrangement, a length of the expandable section may decrease and a diameter of an intermediate portion along a length of the expandable section may increase. One or both of the proximal end and the distal end of the embolic implant may retain their diameters (e.g., the outer diameter of the tube from which the embolic implant is formed, etc.) as the expandable section expands.

The expandable sections of embodiments of embolic implants with expandable sections that self-expand may be shape set to predetermined expanded arrangements. For example, embolic implants formed from shape memory materials may self-expand upon being released from a constraint and exposure to appropriate conditions within a body of a subject (e.g., body temperature, etc.). As another example, implants that are formed from materials with sufficient spring constants may self-expand upon being released from a constraint.

Embodiments of embolic implants with expandable sections that expand under force may have configurations that enable their lengths to be compressed and/or the diameters of their expandable sections to be increased. Appropriate forces may be applied by delivery devices and/or other ancillary devices (e.g., balloon catheters, etc.).

The expandable section of the embolic implant may include an arrangement of cuts, or slits, through a wall of the tube. The slits define struts that extend along at least a portion of the length of the tube. Each strut may have a somewhat rectangular shape, which may enable the strut or portions thereof to occlude a void or passage. The slits may be oriented parallel to one another and in rows in which slits are arranged end-to-end. The rows of slits and the slits of each row may extend helically around the tube. The slits of a row of slits may be offset from the slits of each circumferentially adjacent row of slits. The offsets may be arranged as a so-called “running bond pattern” of slits, in which each slit extends along about half of a length of each circumferentially adjacent slit.

As an expandable section expands to its expanded arrangement, the struts may rotate about their longitudinal axes (i.e., twist).

The membrane of the embolic implant may be positioned over at least a portion of the expandable section of the embolic implant. The membrane may be secured (e.g., adhesively, etc.) to an exterior of the expandable section (e.g., to struts in embodiments where the expandable section includes struts, etc.). The membrane may be formed from a material that enables it to assume a compact configuration as the expandable section is in its unexpanded arrangement (e.g., prior to expansion of the expandable section, etc.) that enables it to expand as the expandable section expands to its expanded arrangement. For example, the membrane may comprise a pliable, inelastic membrane. As another example, the membrane may comprise an elastic membrane.

A shape of the membrane may enable it to collect fluids (e.g., blood, etc.) that flow toward the embolic implant and into the expandable section while in its expanded arrangement. Thus, the membrane may include a primary opening that will face, or oppose, the flow of fluids when the embolic implant is placed within a subject's body, enabling the primary opening to receive the fluid and to be pressurized by the fluid. Without limitation, the primary opening may be located on a proximal side of the membrane. Fluid pressure against the membrane may force the membrane against surfaces of a space (e.g., a vessel, a void, etc.) within which the embolic implant is located, which may help secure the embolic implant in place within the space.

Optionally, the membrane may include one or more secondary openings. Each secondary opening may be positioned on an opposite side of the membrane from the primary opening. Each secondary opening may allow some fluid to flow out of the membrane while maintaining sufficient pressure within the membrane to secure the embolic implant in place within the space where the embolic implant has been positioned. The secondary opening(s) may prevent pressure within the membrane from increasing to a level that will counteract the position-maintaining effect of the membrane by forcing the embolic implant from the space in which it has been placed and positioned.

In another aspect, an embolic system includes a delivery system and an embolic implant. The delivery system may include a wire and a release at a distal end of the wire. The embolic implant may comprise an expandable section and a membrane that covers at least a portion of the expandable section. The membrane may be pressurized in a manner that enable it to apply pressure to surface that define a space within which the embolic implant is placed to at least partially hold the embolic implant in place within the space.

A method for positioning an embolic implant within a body of a subject may include advancing the embolic implant to a desired location, or a target location, within the body of the subject, expanding the expandable section of the embolic implant to an expanded arrangement, and allowing fluid to flow into a primary opening of a membrane over the expandable section and into an interior of the expandable section.

The embolic implant may be advanced and delivered to the desired location (e.g., a blood vessel, another tube, a void, etc.) with a delivery system. The delivery system may release the embolic implant once it is at the desired location and the expandable element has expanded. The delivery system may also be used to reposition the embolic implant (i.e., move the embolic implant from a first location to a second location). In some cases, the delivery system may recapture the embolic implant before repositioning the embolic implant.

As the expandable section of the embolic implant expands, the membrane also expands and defines a boundary between an interior of the expandable section of the embolic implant and an exterior of the expandable section. As fluid flows into the interior of the expandable section, a resulting fluid pressure may enable the membrane to exert pressure against surfaces that define the space in which the embolic implant has been positioned. Such pressure may secure the embolic implant in place within the space where it has been positioned.

Other aspects of the disclosed subject matter, as well as features and advantages of the disclosed subject matter, should become apparent to those of ordinary skill in the art through consideration of the preceding disclosure, the ensuing description, the accompanying drawings, and the appended claims.

### BRIEF DESCRIPTION OF THE DRAWINGS

In the drawings:

FIG. 1 is a perspective view of an embodiment of an embolic implant;

FIG. 2 is an image showing an example of a cut pattern in a tube that may define an embolic implant, such as that depicted by FIG. 1;

FIG. 3 is a perspective view showing the embodiment of embolic implant shown in FIG. 1 with a cover over a portion of an expandable section of the embolic implant;

FIG. 4 is a schematic representation showing a method of introducing the embolic implant of FIG. 3 to a target location in a body of a subject; and

FIG. 5 is a schematic representation showing the embolic implant of FIG. 3 deployed at the target location within the body of the subject.

### DETAILED DESCRIPTION

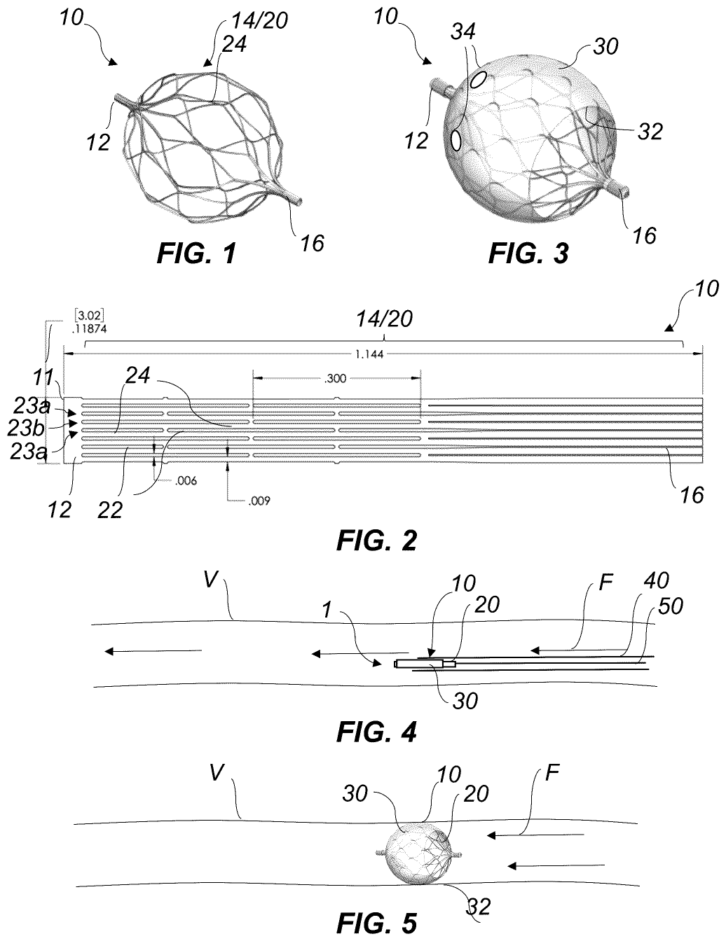

FIG. 1 illustrates an embodiment of an embolic implant 10 in an expanded arrangement. When in the expanded arrangement, the embolic implant 10 may be somewhat spherical in shape. The embolic implant 10 includes a distal end 12, an intermediate section 14, and a proximal end 16.

The intermediate section 14 may be between the distal end 12 and the proximal end 16. The intermediate section 14 of the embolic implant 10 defines an expandable section 20.

The proximal end 16 of the embolic implant 10 may enable the embolic implant 10 to be coupled to a delivery device 50 (FIG. 4) that delivers the embolic implant 10 to a target location (e.g., a location within a body of a subject, etc.). The proximal end 16 may have a configuration that enables the embolic implant 10 to be released by the delivery device 50 once the embolic implant 10 has been advanced to and, optionally, positioned within the target location. In some embodiments, a configuration of the proximal end 16 may also enable the delivery device 50 to reengage the embolic implant 10 to facilitate its repositioning within the target location, movement within the target location or to another location, or removal (e.g., from the body of the subject, etc.).

The embolic implant 10, including its distal end 12, intermediate section 14, and proximal end 16, may be formed from a tube 11 (see FIG. 2, which shows the tube 11 as if it has been cut along its length and flattened). Without limitation, the tube 11 may comprise a hypotube. In some embodiments, the hypotube may be formed from a metal or a metal alloy. For example, the hypotube may be formed from a shape memory material, such as a shape memory alloy (e.g., a nickel-titanium alloy, nitinol, a nickel-chromium-based superallow (e.g., INCONEL® alloy, etc.) etc.). Such a hypotube may have the unexpanded arrangement at a first temperature (e.g., less than 37° C., below body temperature, at room temperature (e.g., about 25° C.), etc.) and assume the expanded arrangement when heated to a second temperature (e.g., body temperature, 37° C., etc.). As another example, the hypotube may be defined from a stainless steel (e.g., am austenitic stainless steel, such as grade 304 stainless steel, grade 316 stainless steel, grade 316L stainless steel, etc.). In other embodiments, the hypotube may be formed from a suitable polymer (e.g., polyether ether ketone (PEEK), polyimide, polytetrafluoroethylene (PTFE), etc.). Materials such as stainless steel and polymers that are not affected by changes from room temperature to body temperature may comprise a material that, when relaxed, may assume the expanded configuration but may be resiliently constrained into the unexpanded configuration (e.g., by a catheter, etc.).

The expandable section 20 of the intermediate section 14 of the embolic implant 10 may be defined by forming a plurality of cuts, or slits 22, through the tube 11. Without limitation, the slits 22 may be formed by laser cutting the tube 11. The slits 22 may be oriented parallel to one another and extend longitudinally along the tube 11. Alternatively, the slits 22 may be arranged helically around the tube (e.g., at an angle of about 10° or less to the longitudinal axis of the tube 11 or at an angle of about 5° or less (e.g., at 6°, 4°, 3°, 2°, 1°, etc.) to the longitudinal axis of the tube 11, etc.).

The slits 22 may be oriented parallel to one another and in rows 23a, 23b in which slits 22 are arranged end-to-end. The slits 22 of a row 23a may be offset from the slits 22 of each circumferentially adjacent row 23b, 23a. The offsets may be arranged as a so-called “running bond pattern” of slits 22, in which each slit 22 extends along about half of a length of each circumferentially adjacent slit 22. The number of rows 23 of slits 22 around the tube 11 contributes to the stiffness of softness of the embolic implant 10. An increase in the number of rows 23 of slits 22 around the circumference of the tube 11 corresponds to an increase in the softness of the embolic implant 10. A softer embolic implant 10 may pack in unpredictable ways that may create better blockage, or occlusion, of a vessel or a void. While FIG. 2 illustrates a specific cut pattern for the tube 11, a wide variety of cut patterns that form embolic implants that may be expanded to somewhat spherical shapes, ellipsoid shapes, and somewhat cylindrical shapes may be used. U.S. Patent Application Publication US 2022/0138693 of Fojtik, the entire disclosure of which is hereby incorporated herein, describes some embodiments of patterns for cutting tubes to form expandable sections of embolic implants and various shapes of embolic implants to which the teachings of this disclosure are applicable.

As an expandable section 20 expands to its expanded arrangement, the struts 24 may rotate about their longitudinal axes (i.e., twist). The expandable section 20 may self-expand or it may expand as a length-compressing force and/or a diameter-expanding force is applied to it. As the expandable section 20 expands from its unexpanded arrangement to its expanded arrangement, as shown in FIG. 1, a length of the expandable section 20 may decrease and a diameter of an intermediate portion along a length of the expandable section 20 may increase. One or both of the proximal end 16 and the distal end 12 of the embolic implant 10 may retain their diameters (e.g., the outer diameter of the tube 11 (FIG. 2) from which the embolic implant 10 is formed, etc.) as the expandable section 20 expands.

As shown in FIG. 3 a membrane 30 of the embolic implant 10 may be positioned over at least a portion of the expandable section 20 (FIG. 1) of the embolic implant 10. The membrane 30 may be secured (e.g., adhesively, etc.) to an exterior of the expandable section 20 (e.g., to struts 24, etc.).

The membrane 30 may be formed from a material that enables it to assume a compact configuration as the expandable section 20 is in its unexpanded arrangement (e.g., prior to expansion of the expandable section, etc.) that enables it to expand as the expandable section 20 expands to its expanded arrangement. For example, the membrane 30 may comprise a pliable, inelastic membrane. Such a membrane 30 may comprise a suitable fabric (e.g., a polytetrafluoroethylene, or PTFE, fabric; etc.). Alternatively, such a membrane may comprise a plastic film. As another example, the membrane 30 may comprise an elastic membrane, which may be formed from a stretch fabric, an elastomeric film, or the like.

A shape of the membrane 30 may enable it to collect fluids (e.g., blood, etc.) that flow toward the embolic implant 10 and into the expandable section 20 (FIG. 2) while in its expanded arrangement. Thus, the membrane 30 may include a primary opening 32 that will face, or oppose, the flow of fluids when the embolic implant 10 is placed within a subject's body, enabling the primary opening 32 to receive the fluid and to be pressurized by the fluid. Without limitation, the primary opening 32 may be located on a proximal side of the membrane 30 (e.g., adjacent to the proximal end 16 of the embolic implant 10, etc.). Fluid pressure against the membrane 30 may force the membrane against surfaces of a space (e.g., a vessel, a void, etc.) within which the embolic implant 10 is located, which may help secure the embolic implant 10 in place within the space.

Optionally, the membrane 30 may include one or more secondary openings 34. Each secondary opening 34 may be positioned on an opposite side of the membrane 30 from the primary opening 32 (e.g., near or adjacent to the distal end 12 of the embolic implant 10, etc.). Each secondary opening 34 may allow some fluid to flow out of the membrane 30 while maintaining sufficient pressure within the membrane 30 to secure the embolic implant 10 in place within the space where the embolic implant 10 has been positioned. The secondary opening(s) 34 may prevent pressure within the membrane 30 from increasing to a level that will counteract the position-maintaining effect of the membrane 30 by forcing the embolic implant 10 from the space in which it has been placed and positioned.

Turning now to FIG. 4, an embodiment of an embolic system 1 is illustrated. The embolic system 1 includes a vascular access device 50 (e.g., a catheter, a cannula, a needle, etc.), a delivery system 50, and an embolic implant 10. The delivery system 50 may include a wire and a release at a distal end of the wire. The embolic implant 10 may comprise an expandable section 20 and a membrane 30 covering a portion of the expandable section 20. The membrane 30 may be formed from a material and have a shape that enables it to be pressurized to hold the embolic implant 10 in place at a desired location, or a target location, within a body of a subject, such as the blood vessel V depicted by FIG. 4.

With continued reference to FIG. 4, a method for positioning an embolic implant 10 within a body of a subject (e.g., within a blood vessel V, within another tubular organ, within a void, or within another space) may include advancing the embolic implant 10 to a desired location, or a target location, within the body of the subject. The embolic implant 10 may be advanced and delivered to the desired location (e.g., a blood vessel V, another tube, a void, etc.) with a delivery system 50. Such delivery may include through a vascular access device 40, such as a catheter, a cannula, a needle, or any other suitable vascular access device. Advancement of the embolic implant 10 may occur in the same direction fluid F (e.g., blood, etc.) flows (e.g., through the blood vessel V, etc.).

Once the embolic implant 10 is advanced to the target location, it may exit a distal end of the vascular access device 40. While at the target location, the expandable section 20 and membrane 30 of the embolic implant 10 may expand to an expanded arrangement, as illustrated by FIG. 5. The delivery system 50 may release the embolic implant 10 once it is at the desired location and the expandable element 20 and membrane 30 have expanded. The delivery system 50 may also be used to reposition the embolic implant 10 (i.e., move the embolic implant 10 from a first location to a second location). In some cases, the delivery system 50 may recapture the embolic implant 10 before repositioning the embolic implant 10.

As the expandable section 20 of the embolic implant 10 expands, the membrane 30 also expands and defines a boundary between an interior of the expandable section 20 of the embolic implant 10 and an exterior of the expandable section 20. Fluid F may then flow into the primary opening 32 of a membrane 30 and into an interior of the expandable section 20. As fluid F fills the interior of the expandable section 20, a resulting fluid pressure within the membrane 30 may enable the membrane 30 to exert pressure against surfaces that define the space in which the embolic implant 10 has been positioned. Such pressure may secure the embolic implant 10 in place within the space where it has been positioned. Any excess pressure generated by the fluid F that flows into membrane 30 may be released through one or more secondary openings 34 in the membrane 30.

Although the disclosure provides many specifics, the specifics should not be construed as limiting the scope of any of the claims, but merely as providing illustrations of some embodiments of elements and features of the disclosed subject matter that fall within the scopes of the claims. Other embodiments of the disclosed subject matter may be devised that are also within the scopes of the claims. Accordingly, the scope of each claim is limited only by its plain language and the legal equivalents thereto.

1. An embolic implant, comprising:
a tube comprising:
a distal end;
a proximal end;
an expandable section between the distal end and the proximal end and having:
a collapsed state in which the expandable section has an intermediate diameter that is substantially the same as a proximal diameter of the proximal end and a distal diameter of the distal end; and
an expanded state in which the proximal diameter of the proximal end and the distal diameter of the distal end remain substantially unchanged while the intermediate diameter of the expandable section increases; and
a membrane covering a distal side of the expandable section, expandable upon expansion of the expandable section to the expanded state, and including at least one opening on a proximal side of the expandable section that allows fluid to flow into the expandable section and against an inner surface of the membrane.

2. The embolic implant of claim 1, wherein the expandable section of the tube includes a plurality of cuts that define a plurality of struts.

3. The embolic implant of claim 2, wherein:
in the collapsed state, circumferentially adjacent struts are positioned adjacent to each other to impart the expandable section with the intermediate diameter that is substantially the same as a proximal diameter of the proximal end and a distal diameter of the distal end; and
in the expanded state, the circumferentially adjacent struts are spaced apart from each other to increase the intermediate diameter of the expandable section.

4. The embolic implant of claim 2, wherein the plurality of struts comprises a plurality of rows of struts extending along a length of the tube, struts of circumferentially adjacent rows of struts being longitudinally offset from each other.

5. The embolic implant of claim 4, wherein the plurality of struts rotate upon expansion of the expandable element.

6. The embolic implant of claim 1, wherein the membrane captures the fluid when the expandable section is in the expanded state.

7. The embolic implant of claim 6, wherein a configuration of the membrane in combination with fluid pressure exerted from the proximal portion toward the distal portion of the expandable section enables the embolic implant to be secured at a desired location along a length of a vessel.

8. The embolic implant of claim 1, wherein the expandable element comprises a shape memory alloy that transitions from the collapsed state to the expanded state upon exposure to body temperature.

9. The embolic implant of claim 1, wherein the membrane includes a plurality of openings on the proximal side of the expandable section.

10. The embolic implant of claim 1, wherein the membrane does not cover a proximal side of the expandable section.

11. The embolic implant of claim 1, wherein the expandable section and the membrane are retractable.

12. The embolic implant of claim 1, further comprising:
a coupler on the proximal end of the tube to facilitate coupling and uncoupling of the embolic implant to a delivery device.

13. The embolic implant of claim 12, wherein the coupler comprises a threaded coupler.

14. An embolic system, comprising:
a delivery system including:
a wire; and
a detachment device at a distal end of the wire; and
an embolic implant according with a proximal end engageable and disengageable by the detachment device of the delivery system, the embolic implant including
a tube comprising:
a distal end;
a proximal end;
an expandable section between the distal end and the proximal end and having:
a collapsed state in which the expandable section has an intermediate diameter that is substantially the same as a proximal diameter of the proximal end and a distal diameter of the distal end; and
an expanded state in which the proximal diameter of the proximal end and the distal diameter of the distal end remain substantially unchanged while the intermediate diameter of the expandable section increases; and
a membrane covering a distal side of the expandable section, expandable upon expansion of the expandable section to the expanded state, and including at least one opening on a proximal side of the expandable section that allows fluid to flow into the expandable section and against an inner surface of the membrane.

15. The embolic system of claim 14, wherein the detachment device engages and disengages a complementary coupler of the embolic implant.

16. The embolic system of claim 15, wherein the detachment device and the coupler are threaded.

17. A method for positioning an embolic implant within a vessel, comprising:
advancing an embolic implant with an expandable section and a membrane on a distal portion of the expandable section to a desired location within the vessel with a delivery system;
expanding the expandable section and the membrane to an expanded state;
allowing a flow to flow into a proximal portion of the expandable section and into an interior of the membrane, a resulting fluid pressure within the interior of the membrane securing the membrane and the expandable section in place along a length of the vessel; and
releasing the embolic implant from the delivery system.

18. The method of claim 17, wherein advancing the embolic implant comprises advancing an embolic implant coupled to a distal end of a delivery wire of the delivery system.

19. The method of claim 18, wherein releasing the embolic implant comprises causing a detachment device of the delivery system at the distal end of the delivery wire to release a coupler on a proximal end of the embolic implant.

20. The method of claim 17, further comprising:
recapturing the embolic implant with the delivery system.

21. The method of claim 20, further comprising:
repositioning the embolic implant to a second location within the vessel; and
redeploying the embolic implant at the second location along the length of the vessel, fluid pressure within the membrane securing the embolic implant in place at the second location.

22. The method of claim 20, further comprising:
removing the embolic implant from the vessel.
